(19) United States
(12) Patent Application Publication
(10) Pub. No.: US 2025/0228477 A1
(43) Pub. Date: Jul. 17, 2025

(54) Tumor Markers

(71) Applicant: LiGalli B.V., Den Haag (NL)

(72) Inventor: Wilhelmus Nicolaas Gerardus Maria DE LAAT, Den Haag (NL)

(21) Appl. No.: 18/699,071

(22) PCT Filed: Oct. 14, 2022
(86) PCT No.: PCT/EP2022/078614

§ 371 Date: Apr. 5, 2024

(30) Foreign Application Priority Data
Oct. 14, 2021 (EP) 21202753.6

(51) Int. Cl. A61B 5/1473; A61B 5/00; A61B 5/145

(52) U.S. Cl. CPC A61B 5/1473; A61B 5/14507; A61B 5/4294; A61B 5/746

(57) ABSTRACT

The present invention relates to a method for determining the presence of a circulating tumor marker in the human body, comprising detecting the tumor marker in the vagina, wherein the tumor marker originates from a part of the body that is not the vagina. In a preferred embodiment, the tumor marker is detected by means of an aptamer. Even more preferably, the aptamer is present in a vaginal ring device. The vaginal ring device suitably comprises a diagnostic device for performing an intravaginal diagnosis or measurement therefor. The method can for example be used in the continuous monitoring of circulating tumor markers in oncologic patients or in the pretreatment and posttreatment follow-up of oncologic patients. The invention further relates to a vaginal diagnostic ring device for performing the method.

The present invention relates to a method and device for the detection of tumor markers.

A tumor marker is anything present in-or produced by-cancer cells. Tumor markers may also be produced by other cells of the body responding to cancer, but also to certain benign conditions. Tumor markers provide information about a cancer, such as the aggressiveness, what kind of treatment it might respond to, whether is is responding to treatment, or whether the cancer starts growing again after treatment.

Tumor markers were traditionally used in the form of proteins or other compounds that are made in higher amounts by cancer cells than by normal cells. They were assayed primarily in the serum of a patient, but also in urine, stool, tumors or other tissues or bodily fluids of patients.

Increasingly however, genomic markers, found in the tumors themselves and in tumor fragments, that are shed into bodily fluids are being used in diagnosis.

Many different genomic tumor markers are in clinical use, totaling a list of over 100 and growing fast. Circulating tumor markers are for example used to:
1) determine the sort and stage of cancer
2) estimate the prognosis
3) detect residual disease
4) detect recurrent disease
5) assess the efficacy of treatment
6) monitor whether the treatment has stopped working.

Tumor markers may also be measured periodically during or after therapy. Such serial measurements, showing how the level of a marker is changing over time, are usually more meaningful than a single measurement. For example a serial decrease in the level of a circulating tumor marker may indicate that the tumor is responding to treatment, a steady low level may indicate that the tumor is in remission, not growing, whereas an increasing level or unchanged level may indicate that the tumor is not responding or growing.

Oncologists do routine follow-ups with tumor markers in patients before, during, and for a long time after therapy. Evidently, the waiting time for a new test and result leads to high anxiety in patients. They also realize that if the sample is for instance taken every 6 months, that after the last sample with a reassuring negative result the tumor could have started growing as of from the next day, obviously leading to higher levels of this tumor marker in a failed monitoring process. More frequent sampling is generally too costly and maybe not efficient.

It is therefore, an object of the present invention to provide a method and device for measuring tumor markers.

This object is achieved by a method wherein a tumor marker originating from a part of the body that is not the vagina is detected in the vagina, in particular in the vaginal wall fluid. This fluid originates from the vaginal wall. It is different from vaginal discharge or vaginal washing. In fact, the vaginal wall fluid comprises transudate, which is fluid that comes out of the blood vessels in the vaginal wall.

The vagina is not routinely used for sampling of tumor markers, except for the presence of gynecological cancers debouching in the vagina e.g. like cervical or endometrial carcinoma.

It was demonstrated before that several biochemical parameters are present in the mucosal wall of the vagina. Those findings include progesterone, estradiol, FSH and glucose. These parameters are measured with a vaginal device as disclosed in EP 3 359 09. A vaginal diagnostic device as described in EP 3 359 099, comprises a diagnostic device for performing an intravaginal diagnosis or measurement therefor. The device further comprises a first rigid member having a first and second end; a second rigid member having a third and fourth end; a first flexible member coupled between the first and third ends; a flexible part coupled between the second and fourth ends; wherein the first flexible member and/or flexible part are configured for allowing the device to be squeezed by bringing the second and fourth ends together thereby transforming a shape of the device from an extended shape to a collapsed shape for allowing the device to be inserted into a vagina of a user at or near the fornix posterior vaginae, said extended shape corresponding to a substantially oval or annular ring shape; wherein at least one of the first flexible member and the flexible part is at least partially elastic such that the device is prebiased to assume the extended shape when no external force is being applied thereto.

In a particularly preferred embodiment of the invention, diagnostic detection of tumor markers is performed by using the vaginal ring as disclosed in EP 3 359 099. This device allows continuous diagnostics over time, data transfer to a smartphone and the programmable insertion of an algorithm automatically warning in case of changes in the level of a compound out of a certain comfort zone. The ring is in particular useful for monitoring patients that have already been treated for cancer to detect a possible recurrence in an early stage. The ring can periodically test the vaginal wall fluid for the presence therein of a marker for the said tumor.

The invention thus also relates to the use of the vaginal ring as disclosed in EP 3 359 099 for measuring tumor markers in the vagina. For this purpose the device is provided with diagnostic means, such as a sensor that can measure a tumor marker that is present in the vaginal fluid, i.e. the transudate of the vaginal mucosa.

In one embodiment, the tumor markers are detected by means of aptamers. Aptamers are oligonucleotide or peptide molecules that bind to a specific target molecule, i.e. the tumor marker.

In one embodiment, the vaginal ring comprises an electrochemical aptamer-based (E-AB) biosensor that is capable of measuring a particular tumor marker. Such E-AB has the ability to generate an electrochemical signal in response to specific target binding in vivo. The signal is measured by a change in Faradaic current passed through an electrode. This current can be measured by the ring and converted to a signal that provides an indication of the amount of tumor marker bound to the aptamer. After a measurement the sensor may be rinsed to be ready for the next measurement. For this the ring may be provided with a reservoir for rinsing liquid. The specificity of the biosensor depends on the aptamer that is used therein.

The invention will be further illustrated in the example that follows and in which it is shown that tumor markers can indeed be detected in the vagina.

### EXAMPLE

The presence of tumor markers in the transudate of the vaginal mucosa was tested. The tests were done by collection of a tiny amount of transudate by striking the mucosa with a swap (Salivette), centrifuging the swap and analyzing the sample with the conventional assay used for the tumor markers tested.

In all cases this was done in healthy female volunteers, except in the case of patient with breast cancer, post treatment, but with recurrence of the tumor growth and positive tumor marker levels in the serum. In this patient the tumor marker CA 15.3 has been identified in the vaginal transudate. In the serum of the patient the result was 200 k (U)/L, the result in the swap was 46 k(U)/L. After obtaining 10 μl of he fluid in the swap, this was diluted 21 times and used in the conventional assay. The detection limit of the assay was 1.5 k[U]/L. The amount of tumor marker in the sample is then 2.17×21=46 k[U]/L. As a comparison, healthy women were tested for this same marker and it was found that the marker CA 15.3 could not be detected.

In addition, a number of other tumor markers were determined in the transudate of healthy women to demonstrate that it is indeed possible to detect various tumor markers in the vagina. The results show that the markers CA125 and CEA can generally be detected in the vaginal wall fluid. However, when the concentration of these markers is elevated this is an indication of the existence of a tumor (He S-M et al., Med Sci Monit, 2011; 17(11): CR618-625).

The indications for which CA125 is used as a marker are ovarian cancer monitoring lung, breast, bowel cancer. For CEA such indications are colorectal, pancreatic, bowel, lung, breastcancer. CA15.3 is a breast cancer marker.

The results of the various tests, done in a similar way, are shown in the table below.

| | | | | Normal value in |
|---|---|---|---|---|
| Volunteer | Tumor | Concentration in | Yield & | plasma of healthy |
| number | marker | vaginal wall fluid | dilution | volunteers |
| 1 | CA125 | ±3000 k[arb′U]/L | Yield 30 μL | <35 k[IU]/L |
| | | | Dilution 1:10 | |
| 1 | CEA | ±7000 ug/L | Yield 30 uL | <4.3 ug/L |
| | | | Dilution 1:10 | |
| 2 | CA125 | ±25500 k[arb′U]/L | Yield 20 uL | <35 k[IU]/L |
| | | | Dilution 1:10 | |
| 2 | CEA | ±58000 ug/L | Yield 20 uL | <4.3 ug/L |
| | | | Dilution 1:10 | |
| 3 | CA125 | ±15000 k[arb′U]/L | Yield 40 uL | <35 k[IU]/L |
| | | | Dilution 1:5 | |
| 3 | CEA | ±22.000 ug/L | Yield 40 uL | <4.3 ug/L |
| | | | Dilution 1:5 | |
| 4 | CA125 | ±13750 k[arb′U]/L | Yield 20 uL | <35 k[IU]/L |
| | | | Dilution 1:10 | |
| 4 | CEA | ±6900 ug/L | Yield 20 uL | <4.3 ug/L |
| | | | Dilution 1:10 | |
| 5 | CEA | ±19000 ug/L | Dilution 1:11 | <4.3 ug/L |
| 5 | CA125 | ±17000 k[arb′U]/L | Dilution 1:11 | <35 k[IU]/L |
| 5 | AFP | Below detection | Dilution 1:11 | <7 k[IU]/L |
| | | level | | |
| 5 | CA15.3 | Below detection | | <35 k[IU]/L |
| | | level | | |
| 6 | CEA | 15330 ug/L | Dilution 1:6 | <4.3 ug/L |
| 6 | CA125 | 16500 k[arb′U]/L | | <35 k[IU]/L |
| 6 | AFP | Below detection | | <7 k[IU]/L |
| | | level | | |
| 6 | CA15.3 | Below detection | | |
| | | level | | |
| 7 | CA15.3 | 46 k[U]/L | Yield 10 uL | Detection limit |
| | | | Dilution 21× | 1.5 k[U]/L |
| | | | 2.17 × 21 = 46 k[U | |

1. Method for determining the presence of a circulating tumor marker in the human body, comprising detecting the tumor marker in the vagina, wherein the tumor marker originates from a part of the body that is not the vagina.

2. Method as claimed in claim 1, wherein the tumor marker is detected by means of an aptamer.

3. Method as claimed in claim 2, wherein the aptamer is present in a vaginal ring device.

4. Method as claimed in claim 3, wherein the vaginal ring device comprises a diagnostic device for performing an intravaginal diagnosis or measurement therefor, wherein the device further comprises a first rigid member having a first and second end; a second rigid member having a third and fourth end; a first flexible member coupled between the first and third ends; a flexible part coupled between the second and fourth ends; wherein the first flexible member and/or flexible part are configured for allowing the device to be squeezed by bringing the second and fourth ends together thereby transforming a shape of the device from an extended shape to a collapsed shape for allowing the device to be inserted into a vagina of a user at or near the fornix posterior vaginae, said extended shape corresponding to a substantially oval or annular ring shape; wherein at least one of the first flexible member and the flexible part is at least partially elastic such that the device is prebiased to assume the extended shape when no external force is being applied thereto.

5. Method as claimed in any one of the claims 1-4, further comprising the step of determining other biochemical parameters in the vagina.

6. Method as claimed in any one of the claims 1-5, for use in the continuous monitoring of circulating tumor markers in oncologic patients.

7. Method as claimed in any one of the claims 1-5, for use in the pretreatment and posttreatment follow-up of oncologic patients.

8. Method as claimed in any one of the claims 1-7, wherein the aptamer is rinsed after detection to be ready for a new detection.

9. Vaginal diagnostic ring device, comprising a diagnostic sensor for performing an intravaginal diagnosis or measurement therefor, wherein the device further comprises a first rigid member having a first and second end; a second rigid member having a third and fourth end; a first flexible member coupled between the first and third ends; a flexible part coupled between the second and fourth ends; wherein the first flexible member and/or flexible part are configured for allowing the device to be squeezed by bringing the second and fourth ends together thereby transforming a shape of the device from an extended shape to a collapsed shape for allowing the device to be inserted into a vagina of a user at or near the fornix posterior vaginae, said extended shape corresponding to a substantially oval or annular ring shape; wherein at least one of the first flexible member and the flexible part is at least partially elastic such that the device is prebiased to assume the extended shape when no external force is being applied thereto, characterized in that the diagnostic device is configured for detecting a tumor marker.

10. Device as claimed in claim 9, wherein tumor marker detection is performed by means of aptamers technique.

11. Device as claimed in claim 10, wherein the diagnostic sensor is an electrochemical aptamer-based biosensor.

12. Device as claimed in any one of the claims 9-11, further comprising means for generating a warning signal in case of rising tumor marker levels.

13. Device as claimed in any one of the claims 10-11, further comprising means for rinsing the aptamer.
